(19) United States
(12) Patent Application Publication
(10) Pub. No.: US 2024/0212795 A1
(43) Pub. Date: Jun. 27, 2024

(54) SYSTEM, METHOD, AND COMPUTER READABLE MEDIUM CONFIGURED TO USE MACHINE LEARNING MODELS TO DETERMINE OPTIMAL SYNTHETIC ROUTES

(71) Applicant: Prepaire Labs Limited, Dublin (IE)

(72) Inventors: Carl Freer, Dublin (IE); Vicente Jorge Ribas, Barcelona (ES)

(21) Appl. No.: 18/600,190

(22) Filed: Mar. 8, 2024

Related U.S. Application Data
(63) Continuation-in-part of application No. 18/244,888, filed on Sep. 11, 2023.
(60) Provisional application No. 63/405,482, filed on Sep. 11, 2022.

(51) Int. Cl. G16C 20/10; G16C 20/70

(52) U.S. Cl. CPC G16C 20/10; G16C 20/70

(57) ABSTRACT

A subject matter of the present invention is system, method, and computer readable medium configured to synthesize a drug product. Single-step organic-chemistry reactions are provided as inputs to a first machine learning model. At least one synthetic route to a product is determined based on an output of the first machine learning model. A score for each of the at least one synthetic route is determined. The at least one synthetic route and its respective score are provided as inputs to a second machine learning model. At least one qualified route is determined based on an output of the second machine learning model.

### CROSS-REFERENCE TO RELATED APPLICATIONS

This application claims the benefit of U.S. Non-Provisional application Ser. No. 18/244,888 filed Sep. 11, 2023 and U.S. Provisional Application No. 63/405,482 filed Sep. 11, 2022, each of which is hereby incorporated by reference in its entirety.

### BACKGROUND OF THE DISCLOSED SUBJECT MATTER

Planning multi-step chemical reactions to synthesize small organic molecules may be a tedious and time-consuming task. For a decade, researchers have developed algorithms that produce blueprints for the sequences of reactions needed to create small organic molecules, such as drugs and related compounds. However, these algorithms have not proven to be efficient, robust, or optimized for determining efficient routes for the synthesis of the small organic molecules.

### SUMMARY OF THE DISCLOSED SUBJECT MATTER

Disclosed herein are methods, systems, and computer program products to address the needs to produce blueprints, such as efficient routes, for the series of reactions needed to synthesize small organic molecules, such as drugs and related compounds. The techniques, described herein, are efficient, robust, and optimized for determining routes for the synthesis of the small organic molecules.

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

To achieve these and other advantages and in accordance with the purpose of the disclosed subject matter, as embodied and broadly described, the disclosed subject matter includes a method of synthesizing a drug product. Single-step organic-chemistry reactions are provided as inputs to a first machine learning model. At least one synthetic route to a product is determined based on an output of the first machine learning model. A score for each of the at least one synthetic route is determined. The at least one synthetic route and its respective score are provided as inputs to a second machine learning model. At least one qualified route is determined based on an output of the second machine learning model.

The disclosed subject matter also includes a system including a computing node comprising a computer readable storage medium having program instructions embodied therewith, the program instructions executable by at least one processor of the computing node to cause the at least one processor to perform a method. Single-step organic-chemistry reactions are provided as inputs to a first machine learning model. At least one synthetic route to a product is determined based on an output of the first machine learning model. A score for each of the at least one synthetic route is determined. The at least one synthetic route and its respective score are provided as inputs to a second machine learning model. At least one qualified route is determined based on an output of the second machine learning model.

The disclosed subject matter also includes a computer program product including a computer readable storage medium having program instructions embodied therewith, the program instructions executable by a processor to cause the processor to perform a method. Single-step organic-chemistry reactions are provided as inputs to a first machine learning model. At least one synthetic route to a product is determined based on an output of the first machine learning model. A score for each of the at least one synthetic route is determined. The at least one synthetic route and its respective score are provided as inputs to a second machine learning model. At least one qualified route is determined based on an output of the second machine learning model.

It is to be understood that both the foregoing general description and the following detailed description are exemplary and are intended to provide further explanation of the disclosed subject matter claimed.

The accompanying drawings, which are incorporated in and constitute part of this specification, are included to illustrate and provide a further understanding of the method and system of the disclosed subject matter. Together with the description, the drawings serve to explain the principles of the disclosed subject matter.

### BRIEF DESCRIPTION OF THE DRAWINGS

A detailed description of various aspects, features, and embodiments of the subject matter described herein is provided with reference to the accompanying drawings, which are briefly described below. The drawings are illustrative and are not necessarily drawn to scale, with some components and features being exaggerated for clarity. The drawings illustrate various aspects and features of the present subject matter and may illustrate one or more embodiment(s) or example(s) of the present subject matter in whole or in part.

FIG. 1A depicts the activation of Favipiravir upon entering a cell, where an example chemical reaction is shown in accordance with the various embodiments of the present disclosure.

FIG. 1B depicts a display of a computer screen as related to drug targets in accordance with the various embodiments of the present disclosure.

FIG. 2A depicts a single-step chemical reaction and a visualization of a series of queries relating to molecule characteristics to determine the success of said reaction in accordance with the various embodiments of the present disclosure.

FIG. 2B depicts a visualization of the neural network in accordance with the various embodiments of the present disclosure.

FIG. 2C depicts a knowledge graph in accordance with the various embodiments of the present disclosure.

FIG. 3 is a flow chart depicting a method of synthesizing a drug product according to various embodiments of the present disclosure.

FIG. 4 depicts a computing node according to various embodiments of the present disclosure.

### DETAILED DESCRIPTION OF THE INVENTION

The PREPAiRE Artificial-intelligence tool needs to have digested nearly every reaction ever performed so it could transform chemistry and help scientists plan multi-step chemical reactions. Since a decade, researchers have already developed a deep learning algo that produces blueprints for the sequences of reactions needed to create small organic molecules, such as drug compounds. Up until now, PREPAIRE has relied on NLP to have conventionally scoured lists of reactions recorded by others, and drawn on their own intuition to work out a step-by-step pathway to make a particular compound.

Our new AI tool must use deep-learning neural networks to imbibe essentially all known single-step organic-chemistry reactions—about 12.4 million of them according to my research. This will enable it to predict the chemical reactions that can be used in any single step. The tool should repeatedly apply these neural networks in planning a multi-step synthesis, deconstructing the desired molecule until it ends up with the available starting reagents.

We want to discover treatments based on the pathogenic profile, using both chemical molecules and natural remedial ingredients. We need to build chemistry to solve the mismatch. The software needs to autonomously design synthetic pathways to structurally diverse targets, including bioactive substances and natural products.

All of these computer-planned routes need to be executed and offer significant yield improvements and cost savings over previous approaches, provide alternatives to patented routes, or produce targets that were not synthesized previously.

The inadequacy of computer programs reflected, among other factors, their limited knowledge base of chemical transformations, their inability to navigate enormous “trees” of synthetic possibilities in an intelligent fashion, and the lack of higher-order logic prescribing how individual steps should be put together to produce viable pathways. At the core of each ˜50,000 rules is a decision tree for double stereo differentiating condensation of esters with aldehydes. The various conditions within the tree specify the range of admissible and also possible (i.e., not only those based on prior literature precedents—as we are currently doing) substituents or atom types. Importantly, all rules account for stereo—and regioselectivity and also for the “context” of the molecule; that is, for groups incompatible with the reaction or those to be protected (for these and other aspects of rule application including electronic and steric effects).

The reaction rules should be the basic “moves” from which the complete synthetic pathways are to be constructed. Because the number of choices at each retrosynthetic step is ˜100 (commensurate with the number of choices at each step in a chess game), the number of possibilities within n steps scales as 100n. To search such an enormous synthetic space intelligent algorithms are needed to truncate and revert from unpromising “branches” and channel the searches toward the most efficient and elegant sequences of steps.

We need to avoid unpromising routes by using numerous heuristics prohibiting unlikely structural motifs, penalizing reactions that are non-selective, or those that would have to proceed through very strained intermediates. The searches should then be guided toward the most feasible solutions by the so-called scoring functions that evaluate: The sets of substrates made at each step and; The sequences of reactions that were used to reach any particular set. Importantly, to enable searches and scoring of substrate sets rather than individual molecules, the bipartite reaction graphs should be transformed into so-called hypergraphs (with “supernodes” combining several individual substance nodes).

The algorithm navigating these hypergraphs takes advantage of higher-order logic (concatenating individual steps into “strategic sequences,” eliminating sequences of steps in which highly reactive groups are dragged along, etc) and terminates when reaching commercially available (currently, whatever we have in our DB from Drugbank etc) or other synthetically popular substrates (ca. 7,000,000 molecules from literature and patents, each with the value of its connectivity within the Network of Chemistry).

Finally, because up to millions of viable pathways can be found for typical targets, dynamic linear programming algorithms need to be used to retrieve pathways that are not only best scoring but also significantly different from each other. In the pathways presented to the user, each substance should be further inspected via built-in molecular mechanics tools, and each reaction needs to come with suggestions for reaction conditions, literature citation(s) illustrating the type of chemistry, information on which groups need to be protected.

The methods, systems, and computer program products described herein use a combination of artificial intelligence (AI) coalesced with proprietary chemical data intelligence to forensically search a wide variety of reputable medical bias databases in order to efficiently synthesize drug products.

The methods, systems, and computer program products described herein learn from graph-based representations of given molecules and uses deep neural networks to generate possible reactant structures that could be used in a synthetic pathway. Its generative power can generate a multitude of new reaction predictions.

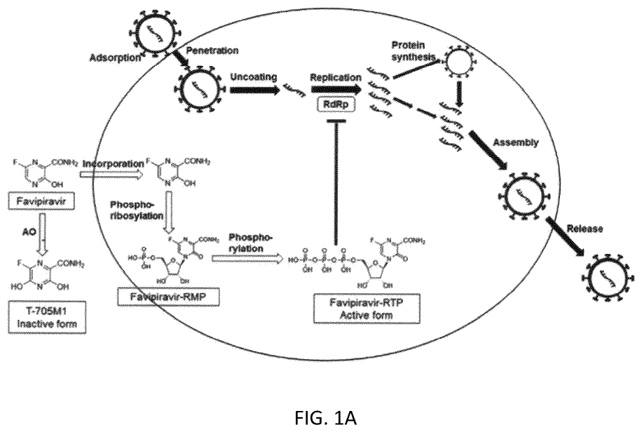

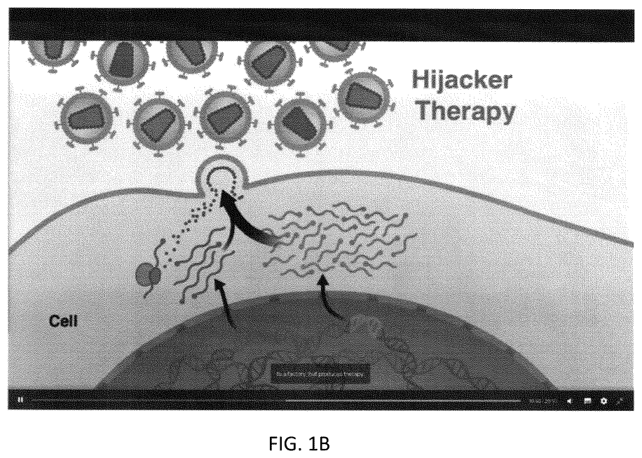

To test the effectiveness of the systems and techniques described herein, a case study was conducted to determine if said systems and techniques could accurately predict the best choice of molecules already in circulation against Covid-19 and Influenza COV2. Favipiravir (depicted in FIG. 1A), a broad spectrum oral anti-viral medication used to treat influenza; and Ivermectin, an anti-inflammatory medication which is used to treat river blindness. Said systems were able to correctly generate a synthesis route for these medicines, and provided alternative combination synthesis routes that suggest a more feasible and effective outcome.

The methods, systems, and computer program products described herein facilitate a hugely intensive search process exploring molecules, determining chemical-reaction-based routes to the molecules, and maintaining the ability to synthesize the molecules (which may or may not be found in nature) using machine learning models. The machine learning models may include the use of one or more neural networks, to imbibe and translate essentially all known single step symbiotic organic chemistry reactions. In particular, the systems and techniques described herein may use one or more machine learning models, such as a generative adversarial network (GAN) and/or a linear programming methodology. A GAN is a machine learning (ML) model in which neural networks compete with each other by using deep learning methods to become more accurate in their predictions. A neural network may include neural programming (including Deep Learning techniques), may be trained using different modes of learning and possibly unstructured and unlabelled data. The use of one or more neural networks may be more suited to linear programming methodology, which may be used in conjunction with the neural network(s).

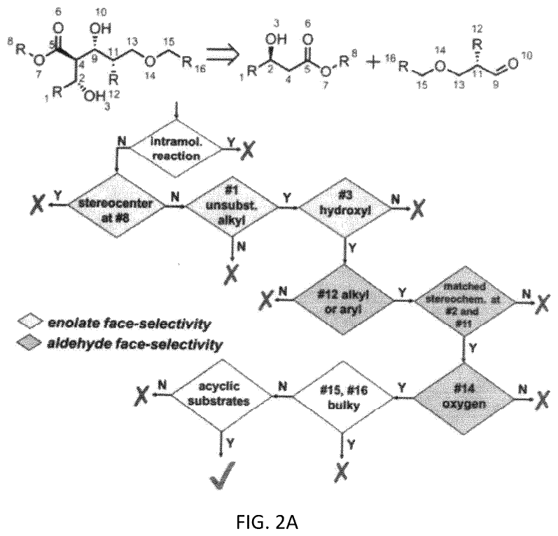

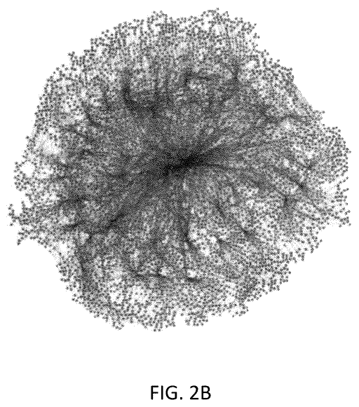

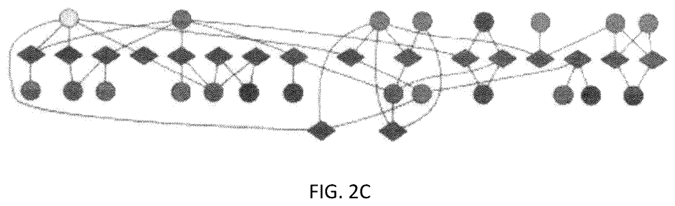

The system described herein is supported by CAS/ACS backed retrosynthesis planning tools and can quickly generate a full retrosynthetic analysis of any known synthetic route(s) for known molecules (FIG. 2A) and assess the interactivity of other molecules alongside the commercial landscape for information regarding reagents. The system allows for the saving and/or publishing of the route(s) for future reference. Many millions of molecules may be assessed, and the system and techniques described herein may be repeatedly evoke neural networks in planning a multi-step routing and synthesis, as well as deconstructing the desired molecule eventuating a summary of available starting materials and reagents (FIG. 2B).

In various embodiments, elements, such as a knowledge graph and/or an AI tool, may be used by the systems and techniques, described herein, to determine optimal synthetic routes to a target drug product. A knowledge graph (KG) (FIG. 2C) may be a data structure that may serve as an illustrative snapshot of research. The KG may be a graph or tree-based data structure, and it may provide a quick, accessible overview of main findings, particularly emphasizing the enhancements to (new data within) a stored database, such as a graph database used by the systems and techniques described herein. The KG may be designed to provide a concise, clear and visual summary of key points and/or determinations as related to drug products. In various embodiments, the KG may be used to determine synthetic routes to a target drug product. In various embodiments, aspects of the KG may be input to, output from, or operated on by the one or more machine learning models described herein.

On an abstract level, the KG may illustrate, with a stylized representation, aspects of a graph database used by the systems and techniques described herein. Displayed as a network graph, the KG may feature various nodes representing the primary elements of a graph database. For example, the primary elements may include variations, proteins, drugs, diseases, pathways, and/or the like. In the KG, the edges connecting the nodes may signify the relationships, such as chemical reaction relationships, between each node. This may form a core facet of the database structure. The KG may include a complex architecture that ultimately can help, process, synthesize, and store comprehensive data in a graph database format. The KG may provide an efficient and user-friendly resource for accessing a wealth of interconnected data. The architecture of a KG may be constructed in several stages:

In an initial stage data may be gathered from various public (or private) databases. These databases may include Crossbar, STRING DB, ClinVar, NCBI, OMIM, MONDO, the database of pharmacophore analysis of active principles (SMILES), DrugBank, and/or the like. Each of these databases may include a different type of data, ranging from genetic variations and associated diseases to drug interactions and protein pathways.

In another stage, the data gathered at the previous stage may be filtered, for example, to determine the most appropriate information for the KG. Once the data is filtered, it may undergo pre-processing to prepare the data for integration into the KG. This stage may include cleaning the data, normalizing it to a standard format, resolving inconsistencies, and dealing with missing values. This pre-processing stage may ensure that the data is in a state suitable for synthesis and integration into the KG.

In yet another stage, the pre-processed data may be analyzed to establish any new connections between different entities. The analysis may involve determining associations between variations and proteins, drugs and diseases, or any other relationships that contribute to the depth and breadth of the knowledge graph. These new connections may be synthesized/determined based on established biological knowledge, computational analysis, and/or the like.

In yet another stage, the synthesized/determined graph connections may be pushed into the graph. These connections, along with the existing nodes and connections of the KG, may be used to determine efficient synthetic routes to a drug product, as described herein. In various embodiments, a third-party knowledge base may be used to corroborate/enhance the architecture of the KG.

The systems and techniques described herein provide a powerful tool to analyze, model, and unravel complex clinical data across a broad range of choices for medical applications.

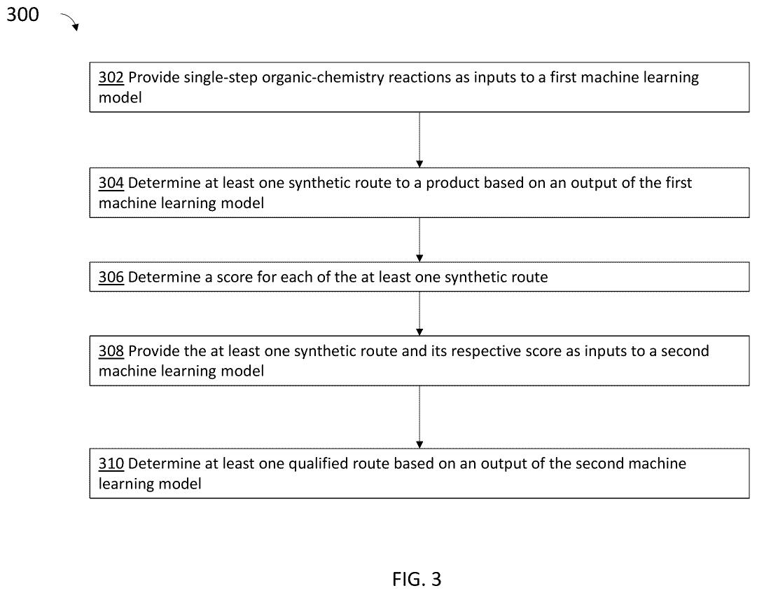

Referring now to FIG. 3, at 302, single-step organic-chemistry reactions are provided as inputs to a first machine learning model. Inputs in addition to or instead of the single-step organic-chemistry reactions may be provided to the first machine learning model, such as information from chemical databases, journals, drug-related information databases, the Internet, and/or the like. The first machine learning model may comprise a neural network model. The neural network model may comprise a generative adversarial network.

At 304, at least one synthetic route to a product is determined based on an output of the first machine learning model. The product may comprise a bioactive substance or a natural product. In various embodiments, the first machine learning model may be able to determine a route to a target drug/product, such as a bioactive substance or a natural product, as well as one or more series of single-step chemical reactions beginning with input elements, chemicals, and/or compounds needed to generate the target drug/product. In various embodiments, a data structure, such as a KG and/or such as a graph or a tree, is used to store information input to and generated by the first machine learning model. For example, the edges of the graph or tree data structure may correspond to a chemical reaction, such as a single-step organic-chemistry reaction, and the nodes of the data structure may correspond to one or more results or compounds created by the chemical reaction. A synthetic route may be a path that may be traversed on such a graph or tree data structure. Such a synthetic route may begin at a node which represents an input, such as an element, chemical, and/or compound, to a chemical reaction, and may terminate at a target drug/product, such as a bioactive substance or a natural product.

At 306, a score for each of the at least one synthetic route is determined. Determining the synthetic route may comprise traversing a tree-based data structure, which may be included in a KG, described herein. In various embodiments, the data structure, such as a graph or a tree, generated at 304, may be traversed. Each path/route that is determined to terminate at a target drug/product, may be assigned one or more numerical values as score(s). These numerical values may be based on one or more factors. For example, the numerical values may be based on: the length of a synthetic route, with a higher (or lower) value for a shorter length, the production cost associated with the reagents and/or reactions on the route, with a higher (or lower) value for a lower production cost, the expected yield of the chemical reactions on the route, with a higher (or lower) value for a higher expected yield, the robustness or efficacy of the resulting target drug/product, with a higher (or lower) value for a greater robustness of efficacy, the toxicity of reagents involved in the synthesis, with a higher (or lower) value for reactions that less toxic reagents, and/or the like. At least one such score may be assigned to each route of the data structure. In various embodiments, the resulting score(s) for each path/route may be normalized to produce a normalized score associated with each path/route.

At 308, the at least one synthetic route and its respective score may be provided as inputs to a second machine learning model. The second machine learning model may comprise a dynamic linear programming model. The second machine learning model may comprise a neural network model. In various embodiments, the data structure generated at 304, and the one or more route scores for each route generated at 306 may be provided as inputs to a second machine learning model, such as a dynamic linear programming model. The second machine learning model may determine one or more of the best, optimized, or highest scoring routes, as well as the corresponding product(s) produced by these routes. For example, the score(s) for each route may be processed, such as by combining the score(s), to determine the one or more best, optimized, or highest scoring routes.

At 310, at least one qualified route is determined based on an output of the second machine learning model. In various embodiments, the second machine learning model may output one or more of the best, optimized, or highest scoring routes as one or more qualified routes. The qualified routes may be determined based on a particular objective determined by a use case, user, entity and/or system to make use of the determined routes. The qualified route(s) may be provided to a user, entity, or system, and may be implemented in a chemical process and/or may be rendered for display on a display of a computing node (FIG. 1A).

In various embodiments, a feature vector that includes the machine learning model inputs may be provided to one or more of the machine learning models described herein. Based on the input features, one or more of the machine learning models described herein may generate one or more outputs. In some embodiments, the output the one or more machine learning models described herein may be a feature vector.

In various embodiments, the one or more machine learning models, described herein, may be pre-trained using training data. In various embodiments training data may be retrospective data. In various embodiments, the retrospective data may be stored in a datastore. In various embodiments, the one or more machine learning models, described herein, may be additionally trained through manual curation of previously generated outputs.

In various embodiments, the one or more machine learning models, described herein, may be and/or may include a dynamic programming algorithm and/or model, such as a dynamic linear programming algorithm/model or a dynamic nonlinear programming algorithm/model. In various embodiments, the one or more machine learning models, described herein, may be a trained classifier. In various embodiments, the trained classifier may be a random decision forest. However, it will be appreciated that a variety of other classifiers are suitable for use according to the present disclosure, including linear classifiers, support vector machines (SVM), or artificial neural network models, such as generative adversarial networks (GANs) and/or recurrent neural networks (RNNs).

Suitable artificial neural network models include but are not limited to a feedforward neural network, a radial basis function network, a self-organizing map, learning vector quantization, a recurrent neural network, a Hopfield network, a Boltzmann machine, an echo state network, long short term memory, a bi-directional recurrent neural network, a hierarchical recurrent neural network, a stochastic neural network, a modular neural network, an associative neural network, a deep neural network, a deep belief network, a convolutional neural networks, a convolutional deep belief network, a large memory storage and retrieval neural network, a deep Boltzmann machine, a deep stacking network, a tensor deep stacking network, a spike and slab restricted Boltzmann machine, a compound hierarchical-deep model, a deep coding network, a multilayer kernel machine, or a deep Q-network.

In various embodiments, the one or more machine learning models described herein may be trained using chemical and/or drug-development data available from public and/or private databases and/or data stores. For example, the machine learning model(s) may be trained to determine all possible routes to a drug product and/or different score(s) for different attributes of any route. As another example, the machine learning model(s) may be trained to determine the best scoring route for any particular objective.

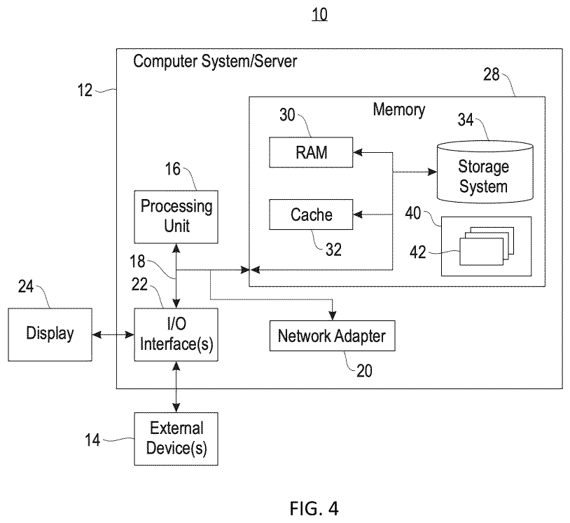

Referring now to FIG. 4, a schematic of an example of a computing node is shown. Computing node 10 is only one example of a suitable computing node and is not intended to suggest any limitation as to the scope of use or functionality of embodiments described herein. Regardless, computing node 10 is capable of being implemented and/or performing any of the functionality set forth hereinabove.

In computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 4, computer system/server 12 in computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, Peripheral Component Interconnect (PCI) bus, Peripheral Component Interconnect Express (PCIe), and Advanced Microcontroller Bus Architecture (AMBA).

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the disclosure.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

In various embodiments, one or more core (not pictured) is coupled to bus 18. In such embodiments, a core may receive data from or write data to memory 28 via bus 18. Likewise, a neurosynaptic core may interact with other components via bus 18 as described herein. In various embodiments, a core may include one or more local controller, memory, or clock, for example as set forth elsewhere herein.

The present disclosure may include a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

While the disclosed subject matter is described herein in terms of certain preferred embodiments, those skilled in the art will recognize that various modifications and improvements may be made to the disclosed subject matter without departing from the scope thereof. Moreover, although individual features of one embodiment of the disclosed subject matter may be discussed herein or shown in the drawings of the one embodiment and not in other embodiments, it should be apparent that individual features of one embodiment may be combined with one or more features of another embodiment or features from a plurality of embodiments.

In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other manners within the scope of the disclosed subject matter such that the disclosed subject matter should be recognized as also specifically directed to other embodiments having any other possible combinations. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents.

Alluding to the above, for purposes of this patent document, the terms “or” and “and” shall mean “and/or” unless stated otherwise or clearly intended otherwise by the context of their use. The term “a” shall mean “one or more” unless stated otherwise or where the use of “one or more” is clearly inappropriate. The terms “comprise,” “comprising,” “include,” and “including” are interchangeable and not intended to be limiting. For example, the term “including” shall be interpreted to mean “including, but not limited to.”

Accordingly, as used herein, terms such as “identifier of an object” and “memory address of an object” should be understood to refer to the identifier (e.g., memory address) itself or to a variable at which a value representing the identifier is stored. As used herein, the term “module” refers to a combination of hardware (e.g., a processor such as an integrated circuit or other circuitry) and software (e.g., machine- or processor-executable instructions, commands, or code such as firmware, programming, or object code).

A combination of hardware and software includes hardware only (i.e., a hardware element with no software elements), software hosted at hardware (e.g., software that is stored at a memory and executed or interpreted at a processor), or at hardware and software hosted at hardware.

Additionally, as used herein, the singular forms “a,” “an,” and “the” include plural referents unless the context clearly dictates otherwise. Thus, for example, the term “module” is intended to mean one or more modules or a combination of modules. Furthermore, as used herein, the term “based on” includes based at least in part on. Thus, a feature that is described as based on some cause, can be based only on that cause, or based on that cause and on one or more other causes.

It will be apparent that multiple embodiments of this disclosure may be practiced without some or all of these specific details. In other instances, well-known process operations have not been described in detail in order not to unnecessarily obscure the present embodiments. The following description of embodiments includes references to the accompanying drawing. The drawing shows illustrations in accordance with example embodiments. These example embodiments, which are also referred to herein as “examples,” are described in enough detail to enable those skilled in the art to practice the present subject matter. The embodiments can be combined, other embodiments can be utilized, or structural, logical and operational changes can be made without departing from the scope of what is claimed. The following detailed description is, therefore, not to be taken in a limiting sense, and the scope is defined by the appended claims and their equivalents.

Alluding to the above, for purposes of this patent document, the terms “or” and “and” shall mean “and/or” unless stated otherwise or clearly intended otherwise by the context of their use. The term “a” shall mean “one or more” unless stated otherwise or where the use of “one or more” is clearly inappropriate. The terms “comprise,” “comprising,” “include,” and “including” are interchangeable and not intended to be limiting. For example, the term “including” shall be interpreted to mean “including, but not limited to.”

The drawing shows illustrations in accordance with example embodiments. These example embodiments, which are also referred to herein as “examples,” are described in enough detail to enable those skilled in the art to practice the present subject matter. The embodiments can be combined, other embodiments can be utilized, or structural, logical and operational changes can be made without departing from the scope of what is claimed. The following detailed description is, therefore, not to be taken in a limiting sense, and the scope is defined by the appended claims and their equivalents.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

The example embodiments of the invention are defined by the following numbered embodiment:

1. A system, method, and computer readable medium configured to use deep learning neutral networks to imbibe essentially all known single step organic chemistry reaction to predict the chemical reaction to be used in any single step as claimed and described herewith.

What is claimed is:

1. A method of synthesizing a drug product, the method comprising:
providing single-step organic-chemistry reactions as inputs to a first machine learning model;
determining at least one synthetic route to a product based on an output of the first machine learning model;
determining a score for each of the at least one synthetic route;
providing the at least one synthetic route and its respective score as inputs to a second machine learning model; and
determining at least one qualified route based on an output of the second machine learning model.

2. The method of claim 1, wherein the first machine learning model comprises a neural network model.

3. The method of claim 2, wherein the neural network model comprises a generative adversarial network.

4. The method of claim 1, wherein the product comprises a bioactive substance or a natural product.

5. The method of claim 1, wherein determining the synthetic route comprises traversing a tree-based data structure.

6. The method of claim 1, wherein the second machine learning model comprises a dynamic linear programming model.

7. The method of claim 1, wherein the second machine learning model comprises a neural network model.

8. A computer program product comprising a computer readable storage medium having program instructions embodied therewith, the program instructions executable by at least one processor to cause the processor to perform a method comprising:
providing single-step organic-chemistry reactions as inputs to a first machine learning model;
determining at least one synthetic route to a product based on an output of the first machine learning model;
determining a score for each of the at least one synthetic route;
providing the at least one synthetic route and its respective score as inputs to a second machine learning model; and
determining at least one qualified route based on an output of the second machine learning model.

9. The computer program product of claim 8, wherein the first machine learning model comprises a neural network model.

10. The computer program product of claim 9, wherein the neural network model comprises a generative adversarial network.

11. The computer program product of claim 8, wherein the product comprises a bioactive substance or a natural product.

12. The computer program product of claim 8, wherein determining the synthetic route comprises traversing a tree-based data structure.

13. The computer program product of claim 8, wherein the second machine learning model comprises a dynamic linear programming model.

14. The computer program product of claim 8, wherein the second machine learning model comprises a neural network model.

15. A system comprising:
a computing node comprising a computer readable storage medium having program instructions embodied therewith, the program instructions executable by at least one processor of the computing node to cause the at least one processor to perform a method comprising:
providing single-step organic-chemistry reactions as inputs to a first machine learning model;
determining at least one synthetic route to a product based on an output of the first machine learning model;
determining a score for each of the at least one synthetic route;
providing the at least one synthetic route and its respective score as inputs to a second machine learning model; and
determining at least one qualified route based on an output of the second machine learning model.

16. The system of claim 15, wherein the first machine learning model comprises a neural network model.

17. The system of claim 16, wherein the neural network model comprises a generative adversarial network.

18. The system of claim 15, wherein the product comprises a bioactive substance or a natural product.

19. The system of claim 15, wherein determining the synthetic route comprises traversing a tree-based data structure.

20. The system of claim 15, wherein the second machine learning model comprises a dynamic linear programming model.
